(19) United States
(12) Patent Application Publication
(10) Pub. No.: US 2015/0144141 A1
(43) Pub. Date: May 28, 2015

(54) Condom Packaging

(71) Applicant: SWIFT! LLC, Memphis, TN (US)

(72) Inventors: Judy Mo, Evanston, IL (US); Samuel W. Malin, Evanston, IL (US); Anne Y. Song, Evanston, IL (US); Xiaorui Xu, Evanston, IL (US)

(73) Assignee: SWIFT! LLC, Memphis, TN (US)

(21) Appl. No.: 14/556,306

(22) PCT Filed: Jun. 10, 2013
(86) PCT No.: PCT/US2013/044916

§ 371 Date: Dec. 1, 2014

Related U.S. Application Data
(60) Provisional application No. 61/658,359, filed on Jun. 11, 2012.

(51) Int. Cl. A61F 6/00; A61F 6/04

(52) U.S. Cl. CPC A61F 6/005; A61F 6/04
USPC 128/844; 206/69

(57) ABSTRACT

Herein is described a method and structure for the extraction of a condom from a condom packet that preserves the orientation of the condom for the user and the correct application position of the condom. The condom is preferably included within a condom packet that includes opposed first and second material layers sealed together along a perimeter defining a sealed cavity between the opposing layers; an exit structure through which a condom can be extracted and delimited by at least three sides of the first material layer; a flap adhesively affixed about at least three sides to the first material layer; and a tab affixed to the flap that can be pulled by a user to cause the flap to peel away from the first material layer and expose the sealed cavity through the exit structure.

This application is a National Stage of PCT/US13/44916, filed 10 Jun., 2013, which claims the priority benefit of U.S. Provisional Patent Application Ser. No. 61/658,359, filed on Jun. 11, 2012, the entire disclosure of which are hereby incorporated by reference.

### FIELD OF THE INVENTION

This invention is related to the storage and use of prophylactics and the process of opening and extracting the prophylactic from the storage container.

### BACKGROUND

The male condom is one of the easiest and cheapest solution for birth control, and the only solution for STI/HIV prevention. When properly used every time users engage in sexual intercourse, condoms are highly effective, with pregnancy rates of approximately 2%. In typical usage, which includes proper and improper uses, pregnancy rates can be as high as 18%.

An estimated 10.4 billion male condoms were used worldwide in 2005 with, approximately, 4.4 billion condoms for family planning and 6.0 billion condoms for HIV prevention. With proper and consistent use, condoms are 98% effective. However, this effectiveness rate for first-year condom users is about 82%, with an estimated rate of only 3% using condoms correctly and consistently during the first year. After this first year period, the condom effectiveness rate increases to around a 96% to 98% rate.

According to a study of 362 men at a first U.S. university, the percentage of users that exhibit the basic incorrect uses are as follows:

| | |
|---|---|
| Not leaving a space at the tip of the condom | 40% |
| Placing the condom upside down on the penis | 30% |
| and then flipping it over | |

Furthermore, 29% of condoms broke and 13% slipped off. Most of these failures occurred after the user exhibited the condoms errors from above. Such problems with condoms are more often a result of errors by the users and rarely from inherent or manufacturing defects of the actual condom, which is why many emphasize the importance and need for sexual health education programs.

In a second survey of 83 undergraduates at a second U.S. university (33 females, 39 males, 11 no gender indicated on response forms), approximately 42.2% of the students felt that applying a condom for the first time is not intuitive. A very large portion, also 42.2%, remembered having difficulty deciding which way to orientate the condom when putting it on at some point in their sexually active lives. Approximately 65% would appreciate an easier way to determine the correct orientation of the condom. Surprisingly, the proportion of women who responded that condom orientation was difficult (48.5%), not intuitive (54.5%), and that they would prefer an easier way (69.7%), suggesting that condom intuitiveness is as much of a consideration for women as it is for men.

Many condom users in the condom survey (n=14) noted that they simply took a close look at the condom while applying it to figure out which way to orientate it. However, pausing a romantic moment to investigate the condom is likely not an ideal solution, especially since there may be no lights on in the room.

Current condom packaging design enables the user to rip the material in many locations around the sides. However, the side opening then requires that the condom be held by its rim as it is removed. This combination of ripping options and removal limitations means that the condom will either be held by the user with an unspecified direction for the reservoir tip. To date, no one has successfully solved the problem of users condom orientation.

### SUMMARY

A first embodiment is a condom packet including opposed first and second material layers sealed together along a perimeter defining a sealed cavity between the opposing layers; an exit structure through which a condom can be extracted and delimited by at least three sides of the first material layer; a flap adhesively affixed about at least three sides to the first material layer; and a tab affixed to the flap that can be pulled by a user to cause the flap to peel away from the first material layer and expose the sealed cavity through the exit structure.

Another embodiment is a packaged condom including a rolled condom, having a body and a tip, enclosed within a sealed cavity; the sealed cavity delimited by a packet that comprises an exit structure that is delimited by at least three sides of a first material layer; a flap covering the exit structure and adhesively affixed to the first material layer; and a tab affixed to the flap that can be pulled by a user to cause the flap to peel away from the first material layer, expose the sealed cavity, and permit the tip of the condom to extend through the exit structure.

Yet another embodiment is a method of extracting a condom from a condom packet including pulling a tab affixed to a flap that covers an exit structure of the condom packet; grasping a tip of the condom which extends through the exit structure; and pulling the condom from the condom packet by the tip, thereby maintaining a user's orientation to an interior and an exterior of the condom while withdrawing the condom from the condom packet.

### BRIEF DESCRIPTION OF THE DRAWINGS

For a more complete understanding of the disclosure, reference should be made to the following detailed description and accompanying drawing figure wherein:

FIG. 1 is a perspective representation of a closed condom packet;

FIG. 2 is a perspective representation of an opened condom packet showing the condom contained therein;

FIG. 3 is a top down representation of a closed condom packet; and

FIG. 4 is a top dorm representation of an opened condom packet showing the condom contained therein.

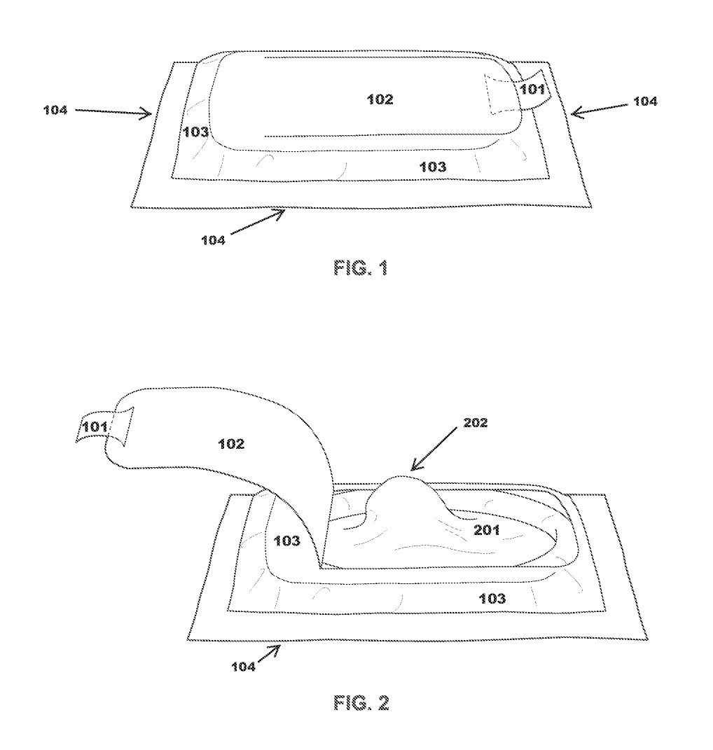

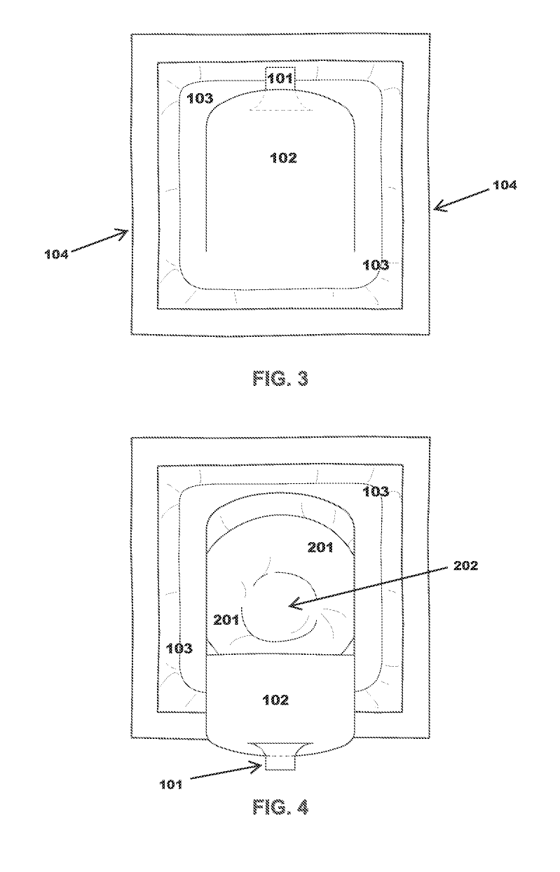

While the disclosed apparatus and method are susceptible to embodiments in various forms, those illustrated in the figure and, hereafter described, in the description of the apparatus and method, are not intended to limit the invention to the specific embodiments described and illustrated herein.

### DETAILED DESCRIPTION

The possibility of the user grasping the condom in the wrong orientation out of the package as addressed herein by the development of a reduced packaging system that 1) requires the package to be opened on one side only and 2) facilitates grasping of the reservoir tip rather than the rim. By providing an opening method that is tactile and intuitive, the number of available opening methods is greatly reduced. With the tab-improved package, the most obvious method of opening is pulling the tab. Once the user has pulled the tab and peeled the top of the package back, the tip of the condom is exposed. While normal condoms are packaged in square packaging, the new design makes use of a rectangular package, which squeezes the condom together and prevents the reservoir tip from folding down and becoming ungraspable.

In reference to the figures, a first embodiment is a condom packet that includes opposed first and second material layers sealed together along a perimeter 104. The sealed perimeter defines a sealed cavity existing between the opposing layers. The condom packet further includes an exit structure through which a condom 201 can be extracted. The exit structure can be delimited by at least three sides of the first material layer 103. The condom packet further includes a flap 102 affixed about at least three sides to the first material layer 103. The flap is preferably adhesively affixed to the three sides of the first material layer. A tab 101 is, preferably, affixed to the flap such that the tab can be pulled by a user to cause the flap 102 to peel away from the first material layer 103 and expose the sealed cavity through the exit structure.

In one example, the flap can be adhesively affixed about at least four sides. In such an example, a user who pulls the tap to cause the flap to peel away from the first material can preferably remove the flap from the condom packet as a single structure.

In another example, the flap is integral to or at least includes a portion of the first material layer. That is, a portion of or the entire flap is seamlessly connected to the first material layer. Preferably, the composition of the flap is the same as the first material layer.

In still another example, the flap is congruent with the exit structure. Preferably, when the flap is congruent with the exit structure the flap is adhesively connected to the first material layer by adhesive tape or one or more overlapping adhesive layers. In an alternative example, the flap is larger than the exit structure. Preferably, three sides of the flap overlap and are adhesively affixed to the first material layer.

The condom packet can have a variety of shapes, for example those selected from square, rectilinear, hexagonal, octagonal, and round. In one preferably example, the condom packet is rectilinear. That is, the condom pack includes four sides, two of which have a length greater than the remaining two. This rectilinear condom packet includes a ratio of the length of the major edge (longer edge) to the length of the minor edge (shorter edge). Preferably, the ratio is in a range of about 1.1 to about 2, more preferably in the range of about 1.1 to about 1.5.

Another embodiment is a packaged condom; that is a rolled condom enclosed within a condom packet. The packaged condom preferably includes a rolled condom 201, having a body and a tip 202, enclosed within a sealed cavity. The sealed cavity delimited by a packet that comprises an exit structure that is delimited by at least three sides of a first material layer 103. The sealed cavity further including a flap 102 covering the exit structure and adhesively affixed to the first material layer; and a tab 101 affixed to the flap that can be pulled by a user to cause the flap to peel away from the first material layer, expose the sealed cavity, and permit the tip 202 of the condom 201 to extend through the exit structure. In one preferable example, the exit structure has an opening of about the same size as a condom thereby providing limited resistance to the removal of the condom from the packet. The packaged condom preferably includes a rectilinear packet, for example those rectilinear packets described above.

The rolled condom enclosed within the condom packet is, preferably, a commercially standard condom and has a circular configuration. The circular configured condom will have an uncompressed diameter determined by the condom manufacturer. In one preferable example, the packaged condom is compressed by the packaging from the circular configuration to an elliptical configuration. For example, the minor edge of the rectilinear packet can have a length that is less than the uncompressed diameter of the condom. In another example, the sealed cavity can have a length or width that is less than the length or width necessary to enclose the condom in an uncompressed, circular configuration. For example, when a standard size condom has a diameter of about two inches the internal length or width of the sealed cavity can be two inches or less.

Yet another embodiment is a method of extracting a condom from a condom packet; that is extracting the condom from the herein described packaged condom. The method of extracting the condom can includes pulling a tab affixed to a flap that covers an exit structure of the condom packet; grasping a tip of the condom which extends through the exit structure; and pulling the condom from the condom packet by the tip. This method maintains a user's orientation to an interior and an exterior of the condom while withdrawing the condom from the condom packet.

Various preferred embodiments of the invention have been described herein. The foregoing description is given for clearness of understanding only, and no unnecessary limitations should be understood therefrom, as modifications within the scope of the invention may be apparent to those having ordinary skill in the art. It should be recognized that these embodiments are merely illustrative of the principles of the present invention. Numerous modifications and adaptations thereof will be readily apparent to those skilled in the art without departing from the spirit and scope of the present invention. Accordingly, the invention is only limited by the claims.

What is claimed:

1. A condom packet comprising:
opposed first and second material layers sealed together along a perimeter defining a sealed cavity between the opposing layers;
an exit structure through which a condom can be extracted and delimited by at least three sides of the first material layer;
a flap adhesively affixed about at least three sides to the first material layer; and
a tab affixed to the flap that can be pulled by a user to cause the flap to peel away from the first material layer and expose the sealed cavity through the exit structure.

2. The condom packet of claim 1, wherein the flap is adhesively affixed about at least four sides.

3. The condom packet of claim 1, wherein the flap comprises a portion of the first material layer.

4. The condom packet of claim 1, wherein the flap is congruent with the exit structure.

5. The condom packet of claim 1, wherein the flap is larger than the exit structure.

6. The condom packet of claim 1, wherein the packet is rectilinear.

7. The condom packet of claim 6, wherein the rectilinear packet includes a ratio of a length of a major edge to a length of a minor edge, wherein the ratio is in a range of about 1.1 to about 2.

8. A packaged condom comprising:
a rolled condom, having a body and a tip, enclosed within a sealed cavity;
the sealed cavity delimited by a packet that comprises an exit structure that is delimited by at least three sides of a first material layer;
a flap covering the exit structure and adhesively affixed to the first material layer; and
a tab affixed to the flap that can be pulled by a user to cause the flap to peel away from the first material layer, expose the sealed cavity, and permit the tip of the condom to extend through the exit structure.

9. The packaged condom of claim 8, wherein the packet is rectilinear.

10. The packaged condom of claim 8, wherein the condom has an uncompressed diameter.

11. The packaged condom of claim 8, wherein the packet has a major edge and a minor edge, and wherein the major edge is longer than the minor edge.

12. The packaged condom of claim 8, wherein the minor edge has a length less than the uncompressed diameter of the condom.

13. The packaged condom of claim 8, wherein the rolled condom enclosed within the sealed cavity is compressed from a circular configuration to an elliptical configuration.

14. A method of extracting a condom from a condom packet comprising:
pulling a tab affixed to a flap that covers an exit structure of the condom packet;
grasping a tip of the condom which extends through the exit structure; and
pulling the condom from the condom packet by the tip, thereby maintaining a user's orientation to an interior and an exterior of the condom while withdrawing the condom from the condom packet.

15. The method of claim 14, wherein pulling the tab uncovers the exit structure and exposes the tip of the condom.
